(19) United States
(12) Patent Application Publication
(10) Pub. No.: US 2005/0070981 A1
(43) Pub. Date: Mar. 31, 2005

(54) Active fixation coronary sinus lead apparatus

(76) Inventor: Sumit Verma, Pensacola, FL (US)

Correspondence Address: S. Michael Bender, P.O. Box 530399, St. Petersburg, FL, 33747, US

(21) Appl. No.: 10/945,418

(22) Filed: Sep. 20, 2004

Related U.S. Application Data
(60) Provisional application No. 60/506,982, filed on Sep. 29, 2003.

(51) Int. Cl. A61F 7/00; A61N 1/05

(52) U.S. Cl. 607/112

(57) ABSTRACT

An active fixation coronary sinus lead apparatus includes electrode support means which include an electrode support longitudinal axis. A plurality of electrode segments are supported by the electrode support means and are arrayed around the electrode support longitudinal axis. A plurality of insulation segments are supported by the electrode support means and are arrayed around the electrode support longitudinal axis at positions opposite to the electrode segments. In this respect, the insulation segments are interspersed between the electrode segments. Electrode segment orientation means are connected to the electrode support means for selectively orienting the electrode segments and the insulation segments around the electrode support longitudinal axis. Vessel anchoring means, which can include three flexible anchor wing portions, are connected to the electrode support means for anchoring the apparatus to an interior wall of a blood vessel. A pressure monitoring system can be connected to the vessel anchoring means for monitoring pressure inside the anchoring means.

### CROSS-REFERENCE TO RELATED APPLICATION

This application claims priority based upon my copending Provisional Application Ser. No. 60/506,982; filed Sep. 29, 2003, which provisional application hereby is incorporated herein by this reference.

### BACKGROUND OF THE INVENTION

1. Field of the Invention

The present invention relates generally to electrodes inserted in a patient, and, more particularly, to an active fixation coronary sinus lead apparatus especially adapted for insertion into a coronary blood vessel.

2. Description of the Prior Art

Recently, it has been shown that patients with congestive heart failure and left bundle branch block benefit from resynchronization pacing therapy with simultaneous stimulation from the left and right ventricles. This has been shown to help patients feel significantly better and possibly live longer. Placement of the right ventricular lead is relatively easy and the lead technology available includes leads that have an active fixation mechanism i.e. a lead with a screw mechanism that can be advanced to attach to the inside of the heart muscle.

Unfortunately the leads placed on the left ventricle are either placed via thoracotomy making this a high risk procedure in patients who are already very ill. Therefore, the currently preferred approach is to place the leads epicardially via the coronary sinus venous branches. The leads currently available do not have an active fixation mechanism to allow fixation to any part of the vein. They have to be placed in a location most distal in the vein so as to minimize the risk of dislodgement. Occasionally this site has poor pacing characteristics i. e. high thresholds, poor sensing or both. If a position more proximal in the vein has better characteristics, it cannot be accepted as there is no reliable way to fix the lead in that position. The only fixation mechanism available clinically is a passive fixation mechanism involving flexible tines. With this, one still has to accept a distal position because the tines will not fix if the vessel caliber is larger than the tines as is usually the case in a more proximal location. In addition the strength of fixation with tines is frequently weak and tined leads may be difficult or impossible to remove after a certain period of time as scar tissue forms around the tines.

It is not uncommon for leads to dislodge from the position achieved, during a procedure as the supporting guides and wires are being removed. This is not only frustrating for the implanting physician, but prolongs procedure times and exposes patients to extra radiation and possible harm because the process of implanting the lead has to be repeated. It is also not unusual for the leads to dislodge at a later time after the patient has left the operating room, due to patient movement and motion of the heart. This will require another operation to reposition the leads. It is evident then that a need exists for a lead that can be positioned in the best location for optimal pacing. It should also be so that this lead can be fixed to this location immediately at the time of implant and if desired can be moved to another location. The lead should be able to be fixed in veins of different caliber. In addition the strength of the fixation should be adequate to prevent dislodgement during removal of the supporting guides and wires and after patient resumes ambulation.

When an expandable implantable electrode is employed, the expansion of the implantable electrode should be in a manner that is controlled accurately. The fixation should be in a manner that is safe and does not result in complications i.e. blood flow though the vein should be uninterrupted and perforation of the vein should not result. Once this is achieved the electrode should be able to pace the heart without causing diaphragmatic stimulation. The availability of a electrode with selective sector activation will allow activation of the electrode in direct contact with the cardiac muscle and allow inactivation of electrodes facing the pericardium, so that diaphragmatic stimulation can be eliminated. These considerations have led to several variations on coronary sinus lead body and electrode designs meant for active fixation inside coronary sinus venous branches. These and related devices are disclosed in U.S. Pat. No. Nos. 4,664,120, 4,699,147, 5,170,802, 5,224,491, 5,344,439, 5,411,546, 5,476,498, 5,951,597, 6,178,356, and 6,529,779, all of which are incorporated herein by reference.

More specifically, U.S. Pat. No. 4,664,120 discloses an atrial-ventricular pervenous lead which employs a flexible material which is spread outward by a plunger action that moves on a longitudinal direction. The flexible material spreads outward by, in essence, pinching or compressing the flexible material. The spreading out of the flexible material is not evenly controlled because there is no material behind the flexible material which assists in the spreading out of the material. In this respect, it would be desirable if an atrial-ventricular lead were provided that included an underlying material that assists an outward flexible material to spread out evenly when the outward flexible material is pinched or compressed.

U.S. Pat. No. 4,699,147 discloses an intraventricular multielectrode cardial mapping probe which includes a plurality of wire assemblies which support electrode segments. The wire assemblies are inherently biased to spread apart from one another. A sliding lumen is used to control the degree of separation of the wire assemblies. The outward spreading pressure that is exerted by the spreading electrodes on the inside walls of a blood vessel is very difficult to control. Therefore, such wire assemblies may pose a danger to the blood vessel walls. This is true whether the electrode segments are used for sensing and mapping electrical signals or would be used for coronary pacing. In this respect, it would be desirable if an active fixation coronary sinus lead apparatus provides accurate pressure control of spreading electrodes on the inside walls of blood vessels.

U.S. Pat. No. 5,244,491 titled “Implantable electrode for location within a blood vessel” (Inventors: Mehra, Rahul. Filed Jun. 30, 1992) refers to an expandable electrode mounted on a delivery system. The referenced patent describes an electrode that is meant to be permanently deployed after expansion of a delivery catheter. Subsequently the delivery catheter has to be removed making this a cumbersome method and raising the possibility of lead dislodgement during delivery catheter removal.

Lacking in U.S. Pat. No. 5,244,491 are a number of features that are provided by the present subject invention. That is, the present invention has the advantage that it can be reversibly deployed in a location of choice by means of a reversibly retractable helix mechanism which allows an accurately controlled expansion. It also includes features to allow either immediate or later removal and re-deployment. The present invention also includes a pressure sensing mechanism to minimize risk of venous perforation. Also, the present invention includes a triangulated configuration of the electrode tip to maximize chances of good electrical contact with the wall and allow continuous blood flow around the vein to prevent clotting. These features make the subject invention more useful for clinical use.

U.S. Pat. No. 5,344,439 discloses a catheter with a retractable anchor mechanism. A mechanism that employs what is in essence a lock and a key friction fit is employed for expanding and contracting a flexible member in a direction which is transverse to a longitudinal action of the lock and key mechanism. There is no underlying material that assists an outward flexible material to spread out evenly when the outward flexible material is expanded by the longitudinal action of the lock and key mechanism.

U.S. Pat. No. 5,411,546 (Title: Defibrillation Electrode, Inventors: Bowald, Hirschberg. Filed Dec. 6, 1993) refers to a defibrillation electrode meant for implantation in an intravascular site. Although this patent refers to an expandable electrode head, the method by which expansion is achieved is quite different from the subject invention.

In contrast with the prior art, the subject invention has a unique safety feature, wherein the mechanism used for expansion of the electrode i.e. a fluid filled chamber compressed with a movable helix is a mechanism that allows an accurately controlled expansion. In addition, the fluid filled chamber serves another purpose. It transmits the pressure to the pressure monitoring device to allow safe expansion of the electrode in a delicate venous structure. In addition the submitted invention refers to a pacing coronary sinus electrode and not a defibrillation electrode.

U.S. Pat. No. 5,951,597 discloses a coronary sinus lead having an expandable matrix anchor which employs a fixation member comprised of a water-permeable polymeric material incorporating an osmotically active agent that swells upon absorbing body fluids therein. The fixation agent seems to be cylindrical in shape, and, as a result, completely obstructs fluid flow in the blood vessel in which the fixation member swells. It is noted that when a active fixation coronary sinus lead apparatus is employed, it would be desirable for blood flow to be permitted through a vessel in which the active fixation coronary sinus lead apparatus is anchored.

U.S. Pat. No. 6,178,356 for “Coronary venous lead having fixation mechanism” (Inventors: Chastain, Tockman, Westlund, Liu, Filed March 1999) refers to a fixation method that includes retention structures with a biodegradable coating. Compared to this referenced patent, the subject invention has the advantage that the expandable electrode can be immediately withdrawn and re-expanded in a different location if so desired. This method provides an immediate method to fix the lead in a desired location if the position is otherwise unstable, as the case would be in a proximal position in a large caliber vein.

Also compared to U.S. Pat. No. 6,178,356, the expandable electrode of the subject invention will allow better contact with the tissue since the electrode is in contact with the tissue under pressure and may allow better pacing and sensing characteristics than would be otherwise possible. U.S. Pat. No. 6,178,356 does not disclose retention structures capable of pacing, as provided by the present subject invention.

U.S. Pat. No. 5,951,597 refers to a “Coronary sinus lead having expandable matrix anchor” (Inventors: Westlund, Tockman, Heil. Filed Apr. 14, 1998). The present subject invention has several advantages over U.S. Pat. No. 5,951,597. The present subject invention allows a mechanism wherein the pacing electrode can be reused immediately or at a later date with ease. It also includes a pressure monitoring system to assess the efficacy of the attachment to the vessel wall. In addition the mechanism of the present subject invention allows immediate fixation to the coronary sinus branch wall allowing more stability during the implant procedure, unlike U.S. Pat. No. 5,951,597.

U.S. Pat. No. 6,529,779 discloses an inflatable electrode for temporary pacing. A temporary balloon-type electrode is provided for insertion and temporary fixation in a blood vessel of a heart, for determining a suitable place therein for a subsequently inserted and fixed permanent electrode, has a catheter and an inflatable and deflatable balloon member disposed at a distal end portion of the catheter. The balloon member has at least one radially expandable hollow body. At least one electrode surface contact member is disposed at a peripheral portion of the hollow body, and flow passages are provided for allowing a blood flow to pass the balloon member when inflated. The temporary balloon-type electrode is particularly suited for use in coronary sinus and peripheral coronary veins of the heart.

One problem not addressed by U.S. Pat. No. 6,529,779 is mentioned hereinabove, and that is that an electrode should be able to pace the heart without causing stimulation of the diaphragm, and thereby cause hiccuping. Hiccuping can interfere with the proper positioning of the electrode and the proper application of pacing signals. More specifically, when a plurality of electrodes are arrayed around a longitudinal axis for pacing the heart, one or more of the electrodes may be positioned so that the diaphragm is stimulated. U.S. Pat. No. 6,529,779 does not provide a mechanism for moving one or more pacing electrodes so that the electrodes will not stimulate the diaphragm. In this respect, it would be desirable if an active fixation coronary sinus lead apparatus were provided which has a mechanism for moving one or more pacing electrodes so that the electrodes will not stimulate the diaphragm.

In summary, the prior art is limited in its inability to provide a safe, reliable, reversible, accurate, immediate and easy method to allow fixation of coronary sinus leads within branches that are usually of variable caliber. In contrast, with the present subject invention, the isovolumetric expansion of the electrode allows accurate assessment of the pressure on the vessel wall to prevent perforation. The present subject invention also allows elimination of diaphragmatic stimulation that is seen in some positions where the coronary sinus lead is placed with the selectively activated electrode sectors. The present subject invention, as explained above, has a number of significant advantages over the prior art and can be constructed with relative ease by those familiar with the materials and methods used for construction of pacemaker leads.

The foregoing desired characteristics are provided by the unique active fixation coronary sinus lead apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides an active fixation coronary sinus lead apparatus which includes electrode support means which include an electrode support longitudinal axis. A plurality of electrode segments are supported by the electrode support means and are arrayed around the electrode support longitudinal axis. A plurality of insulation segments are supported by the electrode support means and are arrayed around the electrode support longitudinal axis at positions opposite to the electrode segments. The insulation segments are interspersed between the electrode segments. Electrode segment orientation means are connected to the electrode support means for selectively orienting the energization of the electrode segments around the electrode support longitudinal axis.

The electrode segment orientation means can include a mechanical ratchet for selecting electrode segment orientation around the electrode support longitudinal axis. It is also recalled from above that the electrode segment orientation means can include an electrically based electrical segment selection means for selectively activating electrode segments and for selectively inactivating electrode segments around the electrode support longitudinal axis. In either case, the energization or activation of a selected electrode segment is accomplished.

Preferably, the electrode segments are arrayed around the electrode support longitudinal axis in a triangular array.

Vessel anchoring means are connected to the electrode support means, for anchoring the apparatus to an interior wall of a blood vessel. Preferably, the vessel anchoring means include a flexible anchor winged member, a distal anchor wing support member, and a distal portion of the electrode support means. The flexible anchor wing member, the distal anchor wing support member, and the distal portion of the electrode support means define a fluid filled chamber. A quantity of fluid is contained in the fluid filled chamber.

Anchor wing expansion/contraction means have one end connected to the distal anchor wing support member and have an opposite end extending outward from the electrode support means. The outwardly extending end is a manually rotatable end. The flexible anchor winged member includes plural anchor wing portions. The plural anchor wing portions provide blood flow channels therebetween and permit blood flow therebetween.

Preferably, the flexible anchor winged member includes three anchor wing portions. The three anchor wing portions are arrayed around the electrode support longitudinal axis in a triangular array. Preferably, the fluid filled chamber is isovolumetric when the anchor wing portions are either expanded or contracted.

The distal portion of the electrode support means is in the form of a fixed plate.

Preferably, the anchor wing expansion/contraction means include a manually retractable helix which is connected between the distal anchor wing support member and the outwardly extending manually rotatable end.

Preferably, a pressure monitoring system is connected between the fluid filled chamber and the proximal end of the electrode support means. The pressure monitoring system includes a fluid pressure monitoring tube, wherein a distal end of the fluid pressure monitoring tube is in fluid communication with the fluid filled chamber. A chamber pressure monitoring and display unit is connected to a proximal end of the fluid pressure monitoring tube. The chamber pressure monitoring and display unit monitors and displays fluid pressure in the fluid filled chamber through the fluid pressure monitoring tube.

In accordance with another aspect of the present invention, a method is provided for applying a voltage to a proximal portion of an interior wall of an anatomical structure which also includes a distal interior wall portion. The method includes the steps of:
obtaining an electrode lead apparatus which includes an electrode segment arrayed around an electrode support longitudinal axis and which includes an insulation segment arrayed around the electrode support longitudinal axis at a position opposite to the electrode segment,
positioning the electrode lead apparatus inside the anatomical structure, such that the electrode segment is adjacent to the proximal interior wall portion and located opposite to the distal interior wall portion, and
energizing the electrode segment adjacent to the proximal interior wall portion, such that distal interior wall portion is not energized.

Preferably, with the method, the anatomical structure is a cardiac structure, and the electrode segment is adjacent to a proximal interior wall portion of the cardiac structure and is located opposite to a distal interior wall portion of the cardiac structure. Often, the distal interior wall portion of the cardiac structure is in close proximity to a diaphragm structure.

Preferably, with the method, the following characteristics are also present. The electrode lead apparatus includes a plurality of electrode segments arrayed around an electrode support longitudinal axis and includes a plurality of insulation segments arrayed around the electrode support longitudinal axis at respective positions opposite to the respective electrode segments. The electrode lead apparatus is positioned inside the anatomical structure, such that a selected electrode segment is positioned adjacent to a selected proximal interior wall portion and such that a respective insulation segment is located opposite to the selected electrode segment which is positioned adjacent to the distal interior wall portion. The selected electrode segment adjacent to the selected proximal interior wall portion is energized, and the distal interior wall portion opposite to the selected proximal interior wall portion is not energized.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining a number of preferred embodiments of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved active fixation coronary sinus lead apparatus which has all of the advantages of the prior art and none of the disadvantages.

It is another object of the present invention to provide a new and improved active fixation coronary sinus lead apparatus which may be easily and efficiently manufactured and marketed.

It is a further object of the present invention to provide a new and improved active fixation coronary sinus lead apparatus which is of durable and reliable construction.

An even further object of the present invention is to provide a new and improved active fixation coronary sinus lead apparatus which is susceptible of a low cost of manufacture with regard to both materials and labor, and which accordingly is then susceptible of low prices of sale to the consuming public, thereby making such active fixation coronary sinus lead apparatus available to the buying public.

In view of the above, a number of advantages and objects of the present invention are provided.

A principal object of the present invention is to provide an active fixation coronary sinus lead apparatus that will overcome the deficiencies of the prior art devices.

Another object of the present invention is to provide an active fixation coronary sinus lead apparatus that allows fixation of the electrode immediately to the coronary sinus branch wall.

Another object of the present invention is to provide an active fixation coronary sinus lead apparatus that allows a reversible fixation to the coronary sinus branch wall.

Another object of the present invention is to provide an active fixation coronary sinus lead apparatus that allows fixation in veins of different diameter.

Another object of the present invention is to provide an active fixation coronary sinus lead apparatus that embodies a design that allows reuse of the attachment mechanism in the same patient immediately or even at a later date without loss of efficacy. In this regard, the helix mechanism can be advanced by means of clockwise turns to decrease the distortion in the fluid chamber and allow flattening of the expandable electrode.

Another object of the present invention is to provide an active fixation coronary sinus lead apparatus with a triangulated expandable electrode that is a safety mechanism that allows continuous blood flow through the vein when the electrode is fully deployed. This minimizes the risk of clotting in the vein while maximizing electrode contact with the underlying tissue to achieve better pacing characteristics.

Another object of the present invention is to provide an active fixation coronary sinus lead apparatus that includes a pressure monitoring system that can minimize the risk of perforation of the vein during implantation of the apparatus. In this regard, the fluid chamber expands in an isovolumetric fashion i.e. the volume of the electrode remains unchanged in the expanded and unexpanded position since the degree of retraction of the tip volumetrically equals the degree of expansion of the three electrodes. This operation will result in the pressure remaining unchanged in the fluid chamber unless external forces from the venous wall are present. Hence the pressure measured will accurately reflect the pressure exerted on the venous wall. This will prevent any perforation of the vein as the pressure can be kept below the vein perforation threshold. This value can be obtained in testing in animal models.

Another object of the present invention is to provide an active fixation coronary sinus lead apparatus that includes an internal retractable helix mechanism within an isovolumetric fluid filled chamber to allow deployment of the expandable electrode in an accurate and controlled manner.

Another object of the invention is to provide an active fixation coronary sinus electrode apparatus that has a triangulated expansion system. This is the preferred embodiment; however a similar result may be obtained with a different number of expandable heads of the electrode. The sectors of the expandable electrode can be selectively tested and activated. The purpose of this feature is to allow best pacing characteristics, while eliminating the incidence of diaphragmatic stimulation occasionally seen after placement of coronary sinus leads as the sectors facing the pericardium are inactivated and only the sector facing the epicardium is active.

Another object of the present invention is to provide an active fixation coronary sinus lead apparatus that is more universally functional in today's market than the prior art devices.

It is intended that any other advantages and objects of the present invention that become apparent or obvious from the detailed description or illustrations contained herein are within the scope of the present invention.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a perspective view of a first embodiment of an active fixation coronary sinus lead apparatus with the reversibly expandable electrode in the expanded position.

FIG. 2 is a longitudinal sectional view of the active fixation coronary sinus lead apparatus of FIG. 1 depicting the interior parts of the lead as seen in the non expanded position.

FIG. 3 is a longitudinal sectional view of the active fixation coronary sinus lead apparatus of FIG. 2 depicting the interior parts of the lead in the expanded position.

FIG. 4 is an end view of the active fixation coronary sinus lead apparatus at the level of the reversibly expandable electrode while it is in the expanded position, viewed from the bottom of the apparatus.

FIG. 5 is a view of the expandable electrode in the expanded state within the branches of the coronary sinus to depict ability to fix in venous branches of different caliber.

FIG. 6 is a view of a pressure monitoring and torque device used for retracting the internal helix mechanism and also to monitor the pressure inside the fluid chamber.

FIG. 7 is a view of a second embodiment of the invention in which an active electrode segment is facing heart muscle and in which an inactive portion is facing away the heart muscle and faces a diaphragm or phrenic nerve.

FIG. 8 is a view is an enlarged side view of the distal end of the pacing electrode of FIG. 7.

FIG. 9 is a cross-sectional view of the embodiment of the invention shown in FIG. 8, taken along line 9-9 thereof.

### DESCRIPTION OF THE PREFERRED EMBODIMENT

With reference to the drawings, a new and improved active fixation coronary sinus lead apparatus embodying the principles and concepts of the present invention will be described.

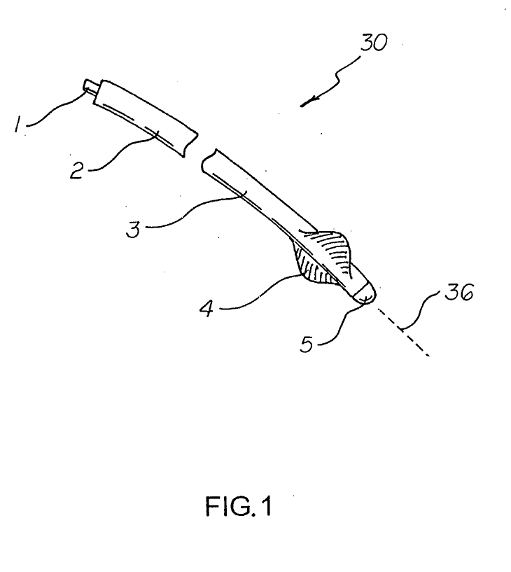

Turning to FIGS. 1-9, there is shown a first embodiment of the active fixation coronary sinus lead apparatus of the invention generally designated by reference numeral 30. In the first embodiment, active fixation coronary sinus lead apparatus 30 is shown with a pressure monitoring and helix torquing mechanism and selectively active electrode sectors for prevention of diaphragmatic stimulation.

Generally, an active fixation coronary sinus lead apparatus 30 includes electrode support means which include an electrode support longitudinal axis 36. A plurality of electrode segments 24 (also called active electrode segments 24) are supported by the electrode support means and are arrayed around the electrode support longitudinal axis 36. A plurality of insulation segments 26 (also called inactive portions 26) are supported by the electrode support means and are arrayed around the electrode support longitudinal axis 36 at positions opposite to the electrode segments 24. The insulation segments 26 are interspersed between the electrode segments 24. Electrode segment orientation means are connected to the electrode support means for selectively orienting the electrode segments 24 around the electrode support longitudinal axis 36.

Preferably, the electrode segments 24 are arrayed around the electrode support longitudinal axis 36 in a triangular array.

Vessel anchoring means are connected to the electrode support means, for anchoring the apparatus to an interior wall of a blood vessel. Preferably, the vessel anchoring means include a flexible anchor winged member, a distal anchor wing support member, and a distal portion of the electrode support means. The flexible anchor wing member, the distal anchor wing support member, and the distal portion of the electrode support means define a fluid filled chamber 10. A quantity of fluid is contained in the fluid filled chamber 10.

Anchor wing expansion/contraction means have one end connected to the distal anchor wing support member and have an opposite end extending outward from the electrode support means. The outwardly extending end is a manually rotatable end. The flexible anchor winged member includes plural anchor wing portions. The plural anchor wing portions provide blood flow channels therebetween and permit blood flow therebetween.

Preferably, the flexible anchor winged member includes three anchor wing portions. The three anchor wing portions are arrayed around the electrode support longitudinal axis 36 in a triangular array. Preferably, the fluid filled chamber 10 is isovolumetric when the anchor wing portions are either expanded or contracted.

The three anchor wing portions and the reversible expandable electrode tips 4 mentioned below are substantially the same. The distal portion of the electrode support means is in the form of a fixed plate 11. The reversible expandable electrode tip 4 mentioned below and the flexible anchor wing member are substantially the same. Also, the distal end of the pacing electrode 5 mentioned below and the distal anchor wing support member are substantially the same.

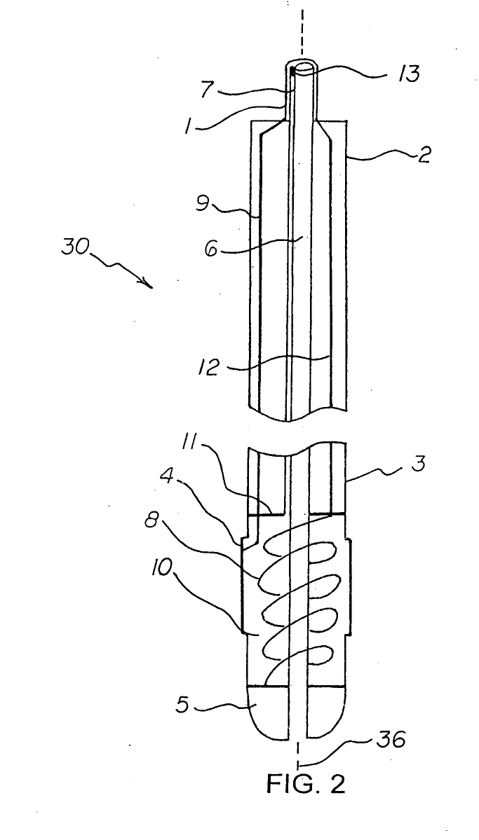

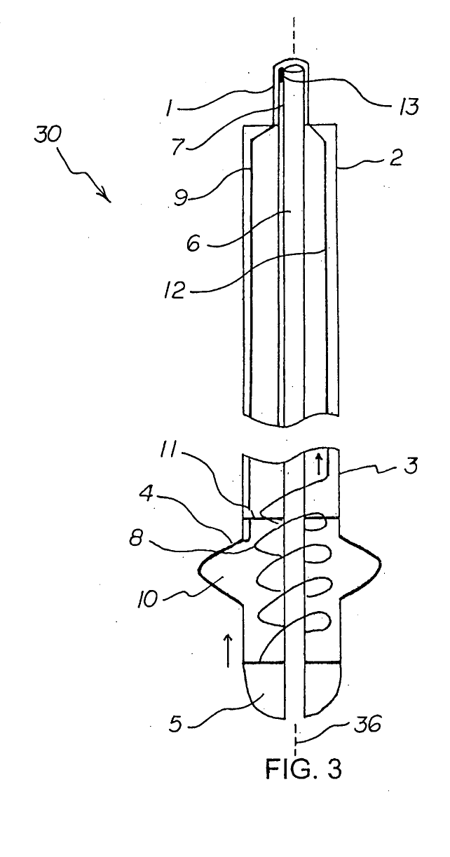

Preferably, the anchor wing expansion/contraction means include a manually retractable helix 8 which is connected between the distal anchor wing support member and the outwardly extending manually rotatable end. Briefly, the anchor wing expansion/contraction means operate as follows. When the outwardly extending manually rotatable end is rotated in one direction, e.g. counterclockwise, the retractable helix 8 pulls up on the distal anchor wing support member causing the distal anchor wing support member to be pulled towards the distal portion 24 of the electrode support means. When this occurs, the quantity of fluid inside the fluid filled chamber 10 is pressurized, and the flexible anchor wing member is spread outward or expanded, as shown in FIG. 3. Conversely, when the outwardly extending manually rotatable end is rotated in the opposite direction, e.g. clockwise, the retractable helix 8 pushes down on the distal anchor wing support member causing the distal anchor wing support member to be pushed away from the distal portion 24 of the electrode support means. When this occurs, the quantity of fluid inside the fluid filled chamber 10 is depressurized, and the flexible anchor wing member is contracted, as shown in FIG. 2.

Preferably, a pressure monitoring system is connected between the fluid filled chamber 10 and the proximal end of the electrode support means. The pressure monitoring system includes a fluid pressure monitoring tube, wherein a distal end of the fluid pressure monitoring tube is in fluid communication with the fluid filled chamber 10. A chamber pressure monitoring and display unit is connected to a proximal end of the fluid pressure monitoring tube. The chamber pressure monitoring and display unit monitors and displays fluid pressure in the fluid filled chamber 10 through the fluid pressure monitoring tube. The fluid pressure monitoring tube is substantially the same as the pressure monitoring channel 7 mentioned below.

More specifically, FIGS. 1, 2, and 3 illustrate an active fixation coronary sinus lead apparatus 30 with a reversibly expandable electrode with a pressure monitoring and helix torquing mechanism and selectively active electrode sectors.

FIG. 1 depicts the electrode in the expanded or deployed state. The proximal end of the pacing electrode is labeled 1. This end will connect to the pacemaker or automatic defibrillator used for pacing through this lead. The proximal end of the pacing electrode 1 can include a mechanical ratchet for selecting electrode segment orientation around the electrode support longitudinal axis 36. Alternatively, the electrode segment orientation means can include an electrically based electrical segment selection means for selecting electrode segments for energization or activation, by which one of the three pacing electrode elements 9 is activated in an insulated manner, so that the other two of the pacing electrode elements 9 can be inactivated.

Reference numeral 2 refers to the proximal insulation covering 2 of the pacing lead which is either poly-urethane or Silicone or similar material. Reference numeral 3 is the distal insulation 3 of the electrode. Reference numeral 4 is the reversible expandable electrode tip 4 which is either a thin malleable metallic filament or a balloon which is coated with conductive material so that this expandable area can function as an active electrode. Reference numeral 5 refers to the distal end of the pacing electrode 5 which also serves as a distal anchor tube support member 5.

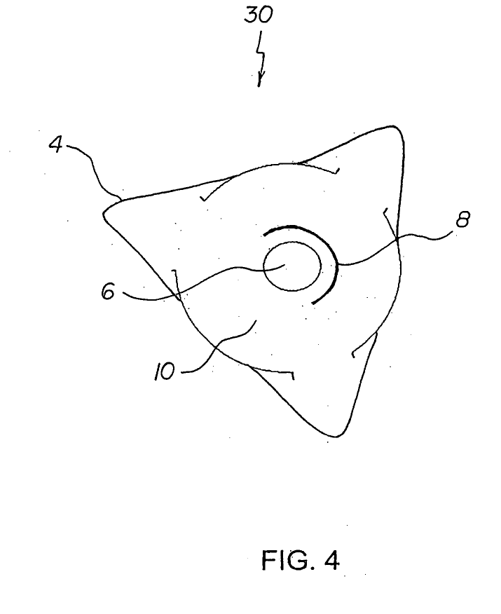

The body of the pacing lead includes several channels as are evident in the drawings especially in the cross-sectional view in FIG. 4. Reference numeral 6 depicts the inner hollow core 6 which allows the lead to be positioned in the coronary sinus branch in an “over the wire” fashion. Reference numeral 7 is the pressure monitoring channel 7 that connects the fluid filled chamber 10 to the proximal end of the lead, so as to allow the pressure to be monitored at the proximal end. Reference numeral 8 is a retractable helix 8 built around the inner hollow core 6. This is connected to the proximal end of the pacing electrode 1.

FIG. 3 depicts the expansion of the expandable electrode. When proximal end of the pacing electrode 1 is turned in a counterclockwise fashion, as depicted in FIG. 3, the retractable helix 8 is retracted into the lead proximally behind the fixed plate 11. The fixed plate 11 forms a boundary for the fluid filled chamber 10. The helix mechanism when retracted behind the proximal fixed plate 11 allows the distal end of the pacing electrode 5 (the distal anchor tube support member) to be approximated to the fixed plate 11 as the helix pulls back the distal end of the pacing electrode 5 (the distal anchor tube support member). This in turn compresses the fluid in the fluid filled chamber 10 and forces expansion of the triangulated reversible expandable electrode tips 4. The degree of expansion of the electrode depends upon the number of counterclockwise turns that are performed at the proximal end of the pacing electrode 1.

Reference numeral 12 represents a torque transmitting member 12 connected from the proximal end of the pacing electrode 1 to the retractable helix 8. The torque transmitting member 12 allows the torque to be transmitted from the proximal end of the pacing electrode 1 to the retractable helix 8. In the drawings, the appearance of the torque transmitting member 12 is simplified and may actually be a helical structure in its entire length.

Reference numeral 13 represents a one-way valve 13 that prevents loss of pressure in the fluid filled chamber 10 but allows monitoring of the pressure when the pressure monitoring and helix torquing mechanism is used.

The selected pacing element 9 is the conductive element of the pacing lead that is selected to connect the distal electrode to the proximal end to allow delivery of sensing signals from the heart and pacing impulses to the heart. The pacing elements 9 include three pacing wires, one from each of the electrode sectors, that are separately insulated so that the three sectors can be selectively paced.

FIG. 4 depicts a cross section of the lead in the distal end in the area of the expandable electrode. This view shows the inner channels encased in the catheter. This includes the inner hollow core 6 and part of the retractable helix 8, which is visible in cross section.

FIG. 4 also shows the reversible expandable electrode tip sectors 4 of the expandable electrode. These reversible expandable electrode tips 4 can be selectively electrically tested and electrically activated. The electrode tip 4 which provides the best contact with the cardiac tissue without causing diaphragmatic stimulation is then selected and activated and the others are inactivated. It is anticipated that this selective activation and deactivation of reversible expandable electrode tips 4 will result in the ability to retain a lead position that would have to be abandoned using the currently available technology which often causes diaphragmatic stimulation. The reversible expandable electrode tip sectors 4 are selected though a ratchet mechanism at the proximal end of the pacing electrode 1.

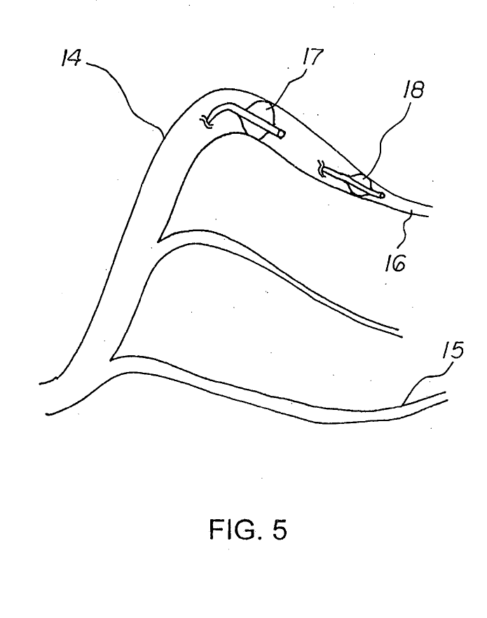

FIG. 5 depicts the coronary sinus anatomy to illustrate the fixation of the active fixation coronary sinus lead apparatus with a reversible expandable electrode in venous branches of different caliber. Reference numeral 14 is the body of the main coronary sinus 14. Reference numeral 15 is the middle cardiac vein 15. Reference numeral 16 is a lateral coronary sinus tributary 16. Reference numeral 17 is the deployed electrode 17 which is deployed in the proximal larger caliber vein. Reference numeral 18 is the deployed electrode 18 which is deployed in a distal narrower part of the same branch. Only the distal end of the active fixation coronary sinus lead apparatus is depicted for simplicity and shows the ability of the lead to be fixed in veins of different calibers simply by different degrees of retraction of the retractable helix 8 to force different degrees of expansion of the expandable electrode 4.

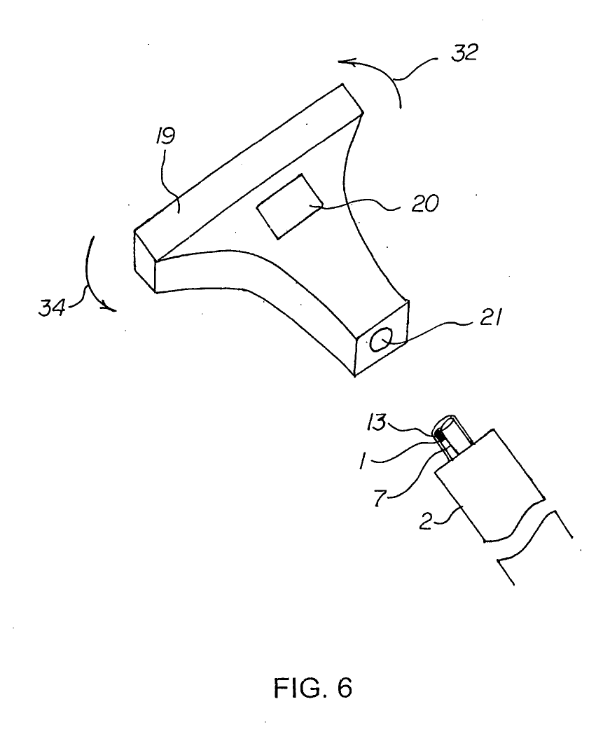

FIG. 6 shows the proximal end 19 of the pressure monitoring and helix torquing mechanism is rotated either clockwise (as shown by directional arrow 32) around the electrode support longitudinal axis 36 or counterclockwise (as shown by directional arrow 34) around the electrode support longitudinal axis 36 for delivery of torque to the retractable helix 8. Reference numeral 20 is a pressure displaying screen 20 preferably a liquid crystal display or a mechanical display. The pressure transducer which drives the pressure displaying screen 20 is encased within the body of this device and is not displayed. Reference numeral 21 is the opening 21 for receiving the proximal end of the pacing electrode 1. The opening 21 connects to the proximal part of the pacing electrode in such a fashion that the valve 13 allows monitoring of the pressure without loss of pressure. The opening 21 also connects to the proximal end of the pacing electrode 1 in a secure manner to allow transmission of the torque to the internal retractable helix 8.

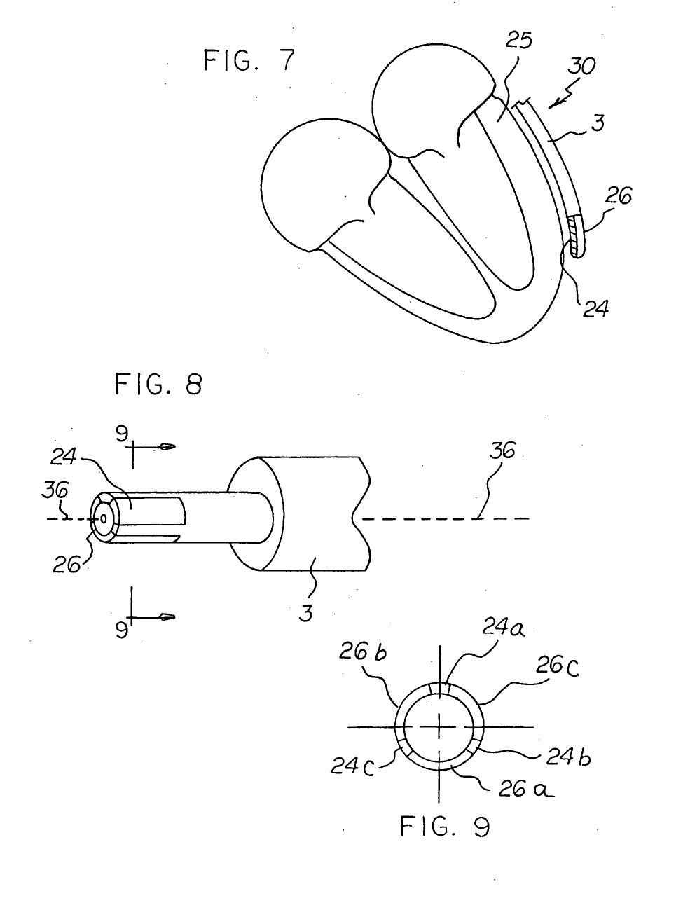

FIGS. 7 to 9 show a second embodiment of the invention in which an active electrode segment 24 is facing heart muscle 25 and in which an inactive portion 26 is facing away the heart muscle 25 and faces a diaphragm (not shown) or phrenic nerve (not shown). The heart muscle 25 adjacent to the active electrode segment 24 is heart muscle of the left ventricle. The body of the active fixation coronary sinus lead apparatus is positioned inside the coronary sinus branch. The active electrode segment 24 touches the heart muscle 25 to pace and sense cardiac contractions. The inactive portion 26 faces the pericardium and touches either the diaphragm or the phrenic nerve.

More specifically, in FIG. 9, active electrode segment 24a is opposite inactive portion 26a; active electrode segment 24b is opposite inactive portion 26b; and active electrode segment 24c is opposite inactive portion 26c. Clearly, with this arrangement of when a respective active electrode segment 24a, 24b, or 24c faces heart muscle 25, the respective opposite inactive portion 26a, 26b, or 26c faces away from the heart muscle 25. Moreover, when a respective active electrode segment 24a, 24b, or 24c faces the heart muscle 25, the respective opposite inactive portion 26a, 26b, or 26c faces toward the diaphragm or the phrenic nerve. As a result, when an active electrode segment 24 faces the heart muscle 25, the diaphragm or phrenic nerve are not stimulated and diaphragmatic stimulation does not occur.

The components of the active fixation coronary sinus lead apparatus of the invention can be made from inexpensive and durable metal and plastic materials.

As to the manner of usage and operation of the instant invention, the same is apparent from the above disclosure, and accordingly, no further discussion relative to the manner of usage and operation need be provided.

It is apparent from the above that the present invention accomplishes all of the objects set forth by providing a new and improved active fixation coronary sinus lead apparatus that is low in cost, relatively simple in design and operation, and which may advantageously be used to implant and fix a pacing lead within the lumen of a coronary sinus vein branch. It embodies an accurate method of expansion of the electrode and a mechanism to monitor the pressure experienced by the electrode tip so as to minimize the risk of blood vessel perforation. An additional benefit of the design is that it does not obstruct blood flow in the expanded state. This invention is more stable in its attachment than other known apparatus, it is safer and it allows a reversible mechanism to attach the electrode. It allows the selective activation of the electrode sectors so that the electrode in direct contact with the cardiac muscle can be activated and others inactivated, so that incidence of diaphragmatic stimulation can be decreased or eliminated. The electrode mechanism is simpler in construction, more universally usable and more versatile in operation than known apparatus of this kind.

A purpose of the present invention is to provide a new reversible active fixation coronary sinus pacing lead device that has many novel features not offered by the prior art apparatus that result method of fixation and safety monitoring which is not apparent, obvious, or suggested, either directly or indirectly by any of the prior art apparatus.

In review, a pacing lead meant for fixation in the coronary sinus to allow pacing of the left ventricle. The apparatus is designed to provide a secure, reversible, accurate, safe and versatile method to attach a coronary sinus pacing lead to the inner aspect of a vein using a variably expandable electrode. The distal end of the electrode includes an internal retractable helix mechanism within a fluid filled chamber. Retraction of the helix is accomplished by counterclockwise turns of the proximal tip of the pacing electrode by methods well known in the prior art. Retraction of the helix compresses the fluid and thereby forces the expandable electrode to expand outward and attach to the vein wall. The electrode expands in an isovolumetric fashion i.e. the volume of the electrode is unchanged in the deployed and undeployed state. Hence the pressure recorded is an accurate measure of stress on the venous wall. The degree of pressure is transmitted through the fluid chamber and through a small channel built into the lead body. It is able to be read at the proximal end of the lead through a pressure transducer system. The pressure transducer is an external device that attaches to the tip of the electrode to allow counterclockwise turns of the lead and to check the pressure. The expandable electrode has a triangular configuration to allow blood flow to continue uninterrupted. One of the three sectors, which provides best pacing characteristics, can be activated and other two are inactivated so as to reduce or eliminate the incidence of diaphragmatic pacing.

Stated somewhat differently, the invention includes an over the wire coronary sinus lead that is either bipolar or unipolar with several modifications described below. The distal end of the lead has an electrode tip. The lead is first introduced to the desired location in the Coronary Sinus Vasculature. Once it is in the desired position, a pressure monitoring system (described in detail above) is attached to the proximal end of the electrode tip and counterclockwise (or clockwise) turns are performed. The pressure monitoring device can measure the pressure inside the fluid filled chamber that lies within an expandable electrode. Also this device can transmit counterclockwise torque to a retractable helix that pulls the distal electrode towards the proximal electrode in a controlled accurate manner. Behind the distal electrode there is a flexible expandable electrode i.e. a conducting metal that is coated onto a flexible expandable surface such as a balloon. This part of the lead includes an inert liquid such as saline that allows the expandable electrode to expand. The expandable electrode when seen in cross section is not circumferential; instead it is triangulated after expansion, this allows continuation of blood flow around the expanded lead body and maximizes chances of good contact with the vessel wall. Since the degree of retraction of the distal tip results in a similar degree of expansion of the expandable electrode sectors, the fluid chamber remains isovolumetric. Hence the pressure remains unchanged, until there is pressure sensed from the venous vessel wall. Thus the pressure sensed is an accurate measure of the stress on the vessel wall. The degree of stress that results in the venous wall perforation can be defined easily in animal studies prior to clinical usage. This should allow minimization of risk of perforation. This will also allow the lead to be secured adequately without expanding the electrode excessively. The attachment mechanism will work by distorting the vessel wall outwardly in a gentle, yet secure fashion. In addition the electrode has a triangulated configuration, and one sector of the electrode can be selectively activated and other three inactivated through a ratchet mechanism at the proximal tip of the electrode.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

1. An active fixation coronary sinus lead apparatus, comprising:
electrode support means which include an electrode support longitudinal axis,
a plurality of electrode segments supported by said electrode support means and arrayed around said electrode support longitudinal axis,
a plurality of insulation segments supported by said electrode support means and arrayed around said electrode support longitudinal axis at positions opposite to said electrode segments, wherein said insulation segments are interspersed between said electrode segments, and
electrode segment orientation means connected to said electrode support means for selectively orienting energization of said electrode segments around said electrode support longitudinal axis.

2. The apparatus of claim 1 wherein said electrode segments are arrayed around said electrode support longitudinal axis in a triangular array.

3. The apparatus of claim 1, further including:
vessel anchoring means, connected to said electrode support means, for anchoring said apparatus to an interior wall of a blood vessel.

4. The apparatus of claim 3 wherein said vessel anchoring means include:
a flexible anchor winged member,
a distal anchor wing support member,
a distal portion of said electrode support means, wherein said flexible anchor wing member, said distal anchor wing support member, and said distal portion of said electrode support means define a fluid filled chamber,
a quantity of fluid contained in said fluid filled chamber,
anchor wing expansion/contraction means having one end connected to said distal anchor wing support member and having an opposite end extending outward from said electrode support means, wherein said outwardly extending end is a manually rotatable end.

5. The apparatus of claim 4 wherein said flexible anchor winged member includes plural anchor wing portions.

6. The apparatus of claim 5 wherein said plural anchor wing portions provide blood flow channels therebetween and permit blood flow therebetween.

7. The apparatus of claim 5 wherein said flexible anchor winged member includes three anchor wing portions.

8. The apparatus of claim 7 wherein said three anchor wing portions are arrayed around said electrode support longitudinal axis in a triangular array.

9. The apparatus of claim 4 wherein said fluid filled chamber is isovolumetric when said anchor wing portions are either expanded or contracted.

10. The apparatus of claim 4 wherein said distal portion of said electrode support means is in the form of a fixed plate.

11. The apparatus of claim 4 wherein said anchor wing expansion/contraction means include a manually retractable helix connected between said distal anchor wing support member and said outwardly extending manually rotatable end.

12. The apparatus of claim 4, further including:
a pressure monitoring system connected between said fluid filled chamber and the proximal end of said electrode support means.

13. The apparatus of claim 12 wherein said pressure monitoring system includes:
a fluid pressure monitoring tube, wherein a distal end of said fluid pressure monitoring tube is in fluid communication with said fluid filled chamber,
a chamber pressure monitoring and display unit connected to a proximal end of said fluid pressure monitoring tube, wherein said chamber pressure monitoring and display unit monitors and display fluid pressure in said fluid filled chamber through said fluid pressure monitoring tube.

14. The apparatus of claim 1 wherein said electrode segment orientation means include a mechanical ratchet for selecting electrode segment orientation around said electrode support longitudinal axis.

15. The apparatus of claim 1 wherein said electrode segment orientation means include an electrically based electrical segment selection means for selectively activating electrode segments and for selectively inactivating electrode segments around said electrode support longitudinal axis.

16. A method for applying a voltage to a proximal portion of an interior wall of an anatomical structure which also includes a distal interior wall portion, comprising the steps of:
obtaining an electrode lead apparatus which includes an electrode segment arrayed around an electrode support longitudinal axis and which includes an insulation segment arrayed around the electrode support longitudinal axis at a position opposite to the electrode segment,
positioning the electrode lead apparatus inside the anatomical structure, such that the electrode segment is adjacent to the proximal interior wall portion and located opposite to the distal interior wall portion, and
energizing the electrode segment adjacent to the proximal interior wall portion, such that distal interior wall portion is not energized.

17. The method of claim 16 wherein:
the anatomical structure is a cardiac structure, and the electrode segment is adjacent to a proximal interior wall portion of the cardiac structure and is located opposite to a distal interior wall portion of the cardiac structure.

18. The method of claim 17 wherein the distal interior wall portion of the cardiac structure is in close proximity to a diaphragm structure.

19. The method of claim 16 wherein:
the electrode lead apparatus which includes a plurality of electrode segments arrayed around an electrode support longitudinal axis and which includes a plurality of insulation segments arrayed around the electrode support longitudinal axis at respective positions opposite to the respective electrode segments,
the electrode lead apparatus is positioned inside the anatomical structure, such that a selected electrode segment is positioned adjacent to a selected proximal interior wall portion and such that a respective insulation segment located opposite to the selected electrode segment is positioned adjacent to a distal interior wall portion,
the selected electrode segment adjacent to the selected proximal interior wall portion is energized, and
the distal interior wall portion opposite to the selected proximal interior wall portion is not energized.
